(19) United States
(12) Patent Application Publication
(10) Pub. No.: US 2024/0299700 A1
(43) Pub. Date: Sep. 12, 2024

(54) CATHETER AND MANUFACTURING METHOD THEREFOR

(71) Applicant: ORBUSNEICH MEDICAL (SHENZHEN) CO., LTD, Shenzhen, Guangdong (CN)

(72) Inventors: Lixia GUO, Shenzhen, Guangdong (CN); YuanLi DONG, Shenzhen, Guangdong (CN); Zhen Yang ZHANG, Shenzhen, Guangdong (CN)

(21) Appl. No.: 18/264,600

(22) PCT Filed: Jan. 10, 2022
(86) PCT No.: PCT/IB2022/050131

§ 371 Date: Aug. 8, 2023

(30) Foreign Application Priority Data
Feb. 9, 2021 (CN) 202110175905.2
Feb. 9, 2021 (CN) 202120359384.1

(51) Int. Cl. A61M 25/00

(52) U.S. Cl. CPC A61M 25/0012; A61M 25/005

(57) ABSTRACT

Disclosing a catheter, comprising a tubular elongated body. The tubular elongated body extends along a longitudinal axis between a proximal end and a distal end. The tubular elongated body has a lumen. At least a portion of the tubular elongated body is defined by a first coil. The first coil comprises a plurality of windings extending along and around the longitudinal axis. The first coil comprises at least one first region arranged near the distal end of the tubular elongated body and including a plurality of adjacent windings. The first region has a plurality of windings inserted from at least one second coil therein. The inserted windings of the second coil are positioned between the windings of the first coil; and the first coil further comprises at least one second region including a plurality of adjacent windings. The second region does not have windings inserted from the second coil.

### TECHNICAL FIELD

The present disclosure relates to a medical device and an apparatus, and in particular to a catheter or microcatheter, and a manufacturing method therefor.

### BACKGROUND ART

A catheter is a thin and flexible tube used in an interventional operation and used for assisting in advancing a therapeutic apparatus to a target site in a body. For example, during the application of percutaneous coronary intervention (PCI) for chronic total occlusion (CTO), a catheter may be used in conjunction with a guide wire to advance a stent into a narrowed portion of a blood vessel and to form a channel for the guide wire to pass through.

With regard to a current interventional catheter, a distal segment thereof is generally supported by a coil layer and/or a braid layer that has a relatively high flexibility to improve the operability of the catheter and to avoid damage to a vessel wall. However, in some cases, the flexibility of the distal segment makes pushability poor during a surgery using such apparatus, and it may be difficult to push the flexible distal segment for advancing in some blood vessels, making it difficult for the catheter to accurately reach a target site or increasing the time of a surgical operation. In addition, when the distal catheter segment is stuck in a stenotic lesion area, or under other conditions (such as withdrawing the catheter from the body), a tensile force needs to be applied to the catheter, at this moment, the distal catheter segment with higher flexibility may present negative reactions under the effect of the tensile force, such as tensile deformation, unwinding or bending, bringing the risk that the guide wire may be bent or kinked.

Accordingly, there is a need to provide a catheter that provides better support and greater passing performance, in order to overcome or partially overcome the above disadvantages and to provide an alternative option other than the existing products.

### SUMMARY OF THE INVENTION

The features and advantages of the disclosure will be illustrated in the following description, part of which will be obvious in the description, or may be learned by practising the principles disclosed herein. The features and advantages of the disclosure may be achieved and obtained by virtue of the means and combinations particularly mentioned in the appended claims.

According to a first aspect of the present disclosure, a catheter is provided, comprising a tubular elongated body, wherein the tubular elongated body extends along a longitudinal axis between a proximal end and a distal end, and the tubular elongated body has a lumen. At least a portion of the tubular elongated body is defined by a first coil, and the first coil comprises a plurality of windings extending along and around the longitudinal axis. The first coil comprises at least one first region arranged near the distal end of the tubular elongated body and including a plurality of adjacent windings, wherein the first region has a plurality of windings inserted from at least one second coil therein, and the inserted windings of the second coil are positioned between the windings of the first coil; and the first coil further comprises at least one second region including a plurality of adjacent windings, wherein the second region does not have windings inserted from the second coil.

According to a second aspect of the present disclosure, a method for manufacturing an elongated catheter is provided, wherein the elongated catheter has a longitudinal axis extending between a proximal end and a distal end and has a lumen. The method comprises: arranging a first end of a first coil near the proximal end of the catheter, and arranging a second end of the first coil near the distal end of the catheter; providing a second coil; and positioning at least some of windings of the second coil in a region of the first coil including a plurality of adjacent coils by screwing a winding at one end of the second coil into the first coil along the longitudinal axis from the second end of the first coil.

### BRIEF DESCRIPTION OF THE DRAWINGS

In order to describe the manner in which the above-mentioned and other advantages and features of the disclosure can be obtained, the principle briefly described above will be more specifically described by reference to specific embodiments illustrated in the accompanying drawings. It should be understood that these drawings depict only exemplary embodiments of the disclosure and are therefore not to be considered limiting of the scope thereof, and the principle herein is described and explained with particular examples and details by using the accompanying drawings. Preferred embodiments of the present disclosure will be described below in further detail, by way of example and with reference to the accompanying drawings, in which:

FIG. 1A shows a schematic diagram of an exemplary catheter.

FIG. 1B shows a schematic diagram of a portion of internal structure of an exemplary catheter.

FIG. 1C shows a schematic structural diagram of a cross section of a distal segment of the exemplary catheter in FIG. 1B.

FIG. 2A shows a schematic structural diagram of a combination of two coils according to an exemplary embodiment.

FIG. 2B shows a schematic diagram of a longitudinal cross section of a distal catheter segment including the combined coils shown in FIG. 2A, wherein the coils are made of a round wire.

FIG. 2C shows a schematic diagram of the longitudinal cross section of the distal catheter segment including the combined coils shown in FIG. 2A, wherein the coils are made of a flat wire.

FIG. 3A shows a schematic structural diagram of a combination of two coils according to another exemplary embodiment.

FIG. 3B shows a schematic diagram of a longitudinal cross section of a distal catheter segment including the combined coils shown in FIG. 3A.

FIG. 4A shows a schematic structural diagram of a combination of three coils according to an exemplary embodiment.

FIG. 4B shows a schematic diagram of a longitudinal cross section of a distal catheter segment including the combined coils shown in FIG. 4A.

FIG. 4C shows a perspective view of the combined coils of the embodiment in FIG. 4A.

FIG. 5A shows a schematic structural diagram of a combination of four coils according to an exemplary embodiment.

FIG. 5B shows a schematic diagram of a longitudinal cross section of a distal catheter segment including the combined coils shown in FIG. 5A.

### DETAILED DESCRIPTION OF EMBODIMENTS

Various embodiments of the present disclosure will be discussed in detail below. While specific implementations are discussed, it should be understood that it is intended for illustrative purposes only. Those skilled in the relevant art will recognize that other components and configurations may be used without departing from the spirit and scope of the present disclosure.

In the following description, the same reference numerals denote the same components. The term “proximal” means a direction close to an operator when a catheter is in use, the term “distal” means a direction away from the operator when the catheter is in use, and the term “longitudinal axis direction” means a direction extending from a proximal end to a distal end along a longitudinal axis. Numerals such as “first”, “second”, “third”, “fourth”, “fifth” and the like in this description are used merely to denote different components/parts, rather than to limit these components/parts.

The present disclosure provides a catheter having a distal segment formed by combining two or more coils. On the premise of providing flexibility without affecting the operability of the catheter, a distal catheter segment has varying strength as desired, thereby improving the pushability of the catheter in a blood vessel and preventing the catheter from deforming under a stress.

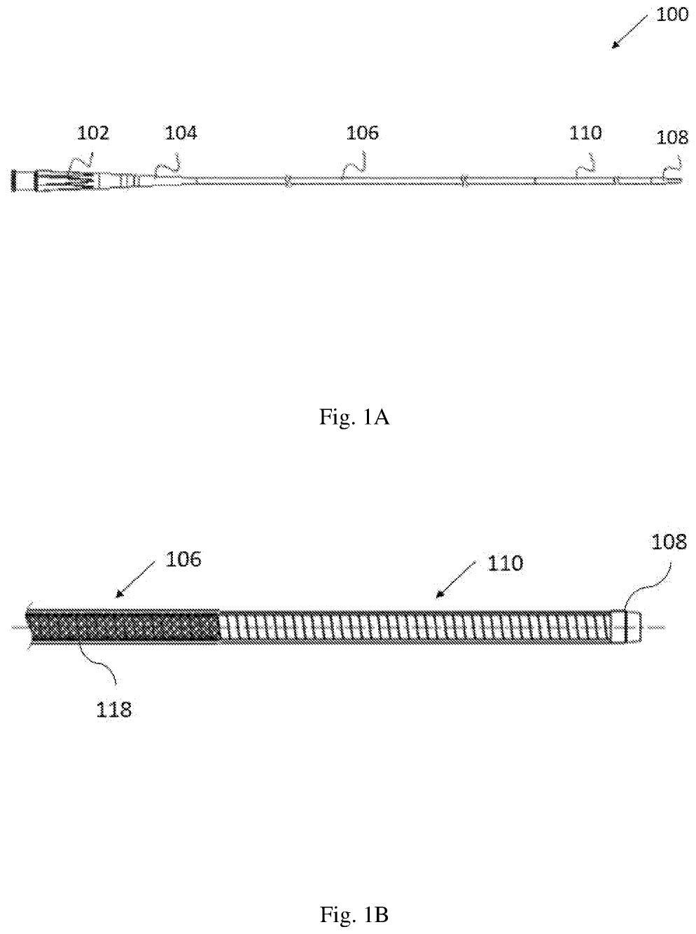

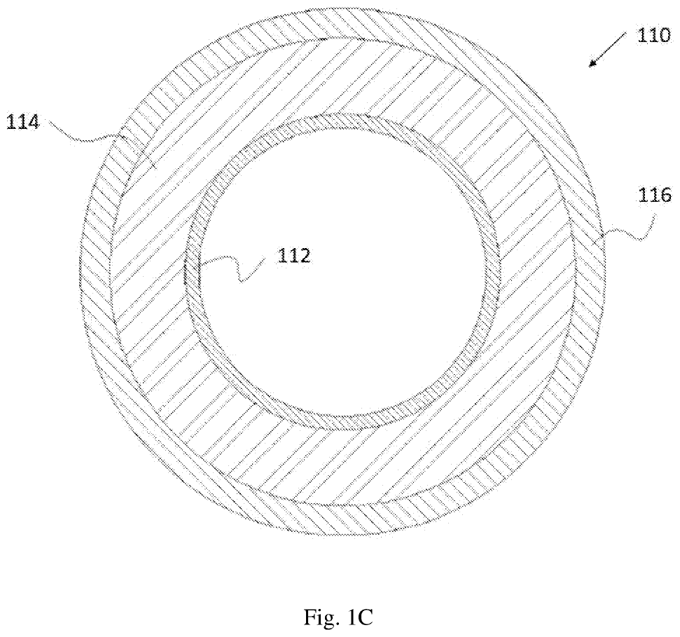

FIG. 1A illustrates an exemplary catheter 100. Referring to FIG. 1, the catheter 100 comprises a hub 102, a connecting member 104, a catheter body and a tip 108, wherein the catheter body comprises a proximal segment 106 and a distal segment 110. The hub 102 is located at a proximal end of the catheter, and the connecting member 104 is disposed between the hub 102 and the catheter body for soft/hard transition between the hub 102 and the catheter body. The catheter body is a tubular elongated body extending along a longitudinal axis between a proximal end and a distal end of the catheter, the interior of the catheter has a lumen for providing a passage for a guide wire, a medicine or a surgical tool. The tip 108 is disposed at the distal end of the catheter body and has sufficient flexibility and toughness to prevent damage to an inner liner of a blood vessel in contact with the tip.

The catheter body has a proximal segment 106 located near the proximal end of the catheter and a distal segment 110 located near the distal end of the catheter along the longitudinal axis thereof. The stiffness of the proximal segment 106 is greater than that of the distal segment 110 so that the proximal segment 106 provides certain support strength and recoil to the distal segment 110, making the catheter body have sufficient torsional thrust and support strength.

FIG. 1B shows a schematic diagram of a portion of internal structure of an exemplary catheter, and FIG. 1C shows a schematic structural diagram of a cross section of the distal segment of the catheter. As shown in FIGS. 1B and 1C, the distal segment 110 is a concentric three-layer structure and comprises: an inner layer 112 having a hollow lumen, a tubular intermediate layer 114 wrapping the inner layer, and a tubular outer layer 116 wrapping the intermediate layer 114, wherein the intermediate layer 114 may comprise a spring coil. Also, the proximal segment 106 of the catheter may be a concentric four-layer structure, in addition to the inner layer 112, the intermediate layer 114 and the outer layer 116 that extend to the distal segment, a braid layer 118 may be additionally included between the inner layer 112 and the outer layer 116 to provide a greater stiffness than that of the distal segment 110.

In some embodiments, the distal segment 110 of the catheter is disposed at a position about 10-60 mm from the end of the tip 108 and may have an outer diameter range from about 0.2 mm to about 2 mm to achieve better side branch intervention. A portion of the distal catheter segment 110 may be bent at an angle along the longitudinal axis of the catheter body so that when the catheter needs to pass through a branch lesion in a blood vessel, such as a coronary artery, the distal segment may be bent in vitro at a corresponding angle in advance to fit an included angle formed by a coronary artery branch and a main branch, thereby facilitating smooth passage of the catheter through the branch lesion. The hollow lumen is formed in the inner layer of the catheter body and may be used for intervening required materials such as a medicine, a guide wire, a surgical tool, a treatment element and the like.

On one hand, the distal catheter segment 110 needs to have flexibility and bendability to allow the catheter 100 to enter a distal vasculature by manipulating a guide wire; and on the other hand, the distal segment needs to have a certain tensile strength such that the distal segment can be pushed through a stenosis site in the blood vessel when the catheter 100 is advanced, and does not deform under a larger tensile force when the catheter 100 is withdrawn.

In a prior catheter, because the inner layer 112, the outer layer 116, and the intermediate layer 114 of the distal catheter segment 110 each extend out from the proximal segment 106 of the catheter and are made of a continuous and consistent material, the strength of each portion is generally consistent, the catheter can only provide a consistent flexibility or tensile strength, and cannot meet the usage requirements in many cases. Although it is possible to achieve changes in the tensile strength of the distal segment 110 by changing a wire material, a wire thickness and the number of wire strands, pitch of coils and the like of the coils of the intermediate layer 114, by means of these conventional approaches, it is still difficult to allow the distal catheter segment 110 to have a strength that varies as required. In addition to this, these changes may increase material costs, the difficulty of coil winding, or the complexity of a manufacturing process, thereby increasing production costs.

In the present disclosure, by combining two or more second coils with a first coil in at least one region of the distal catheter segment 110, the tensile strength of the region is improved. Moreover, the distal catheter segment also comprises at least one region in which only the first coil is provided and no second coil is combined therewith, such that the distal catheter segment 110 achieves different tensile strengths in different regions.

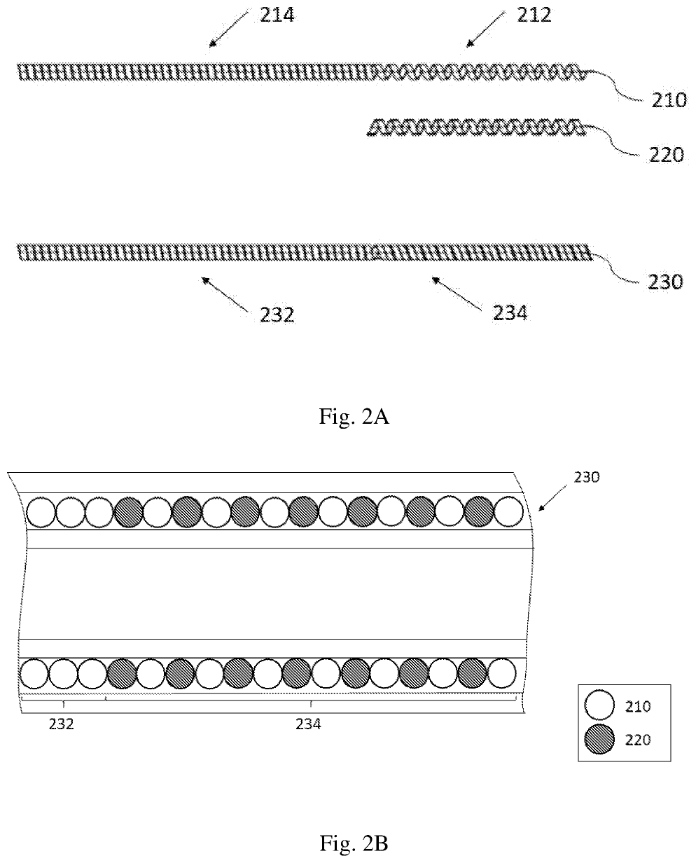

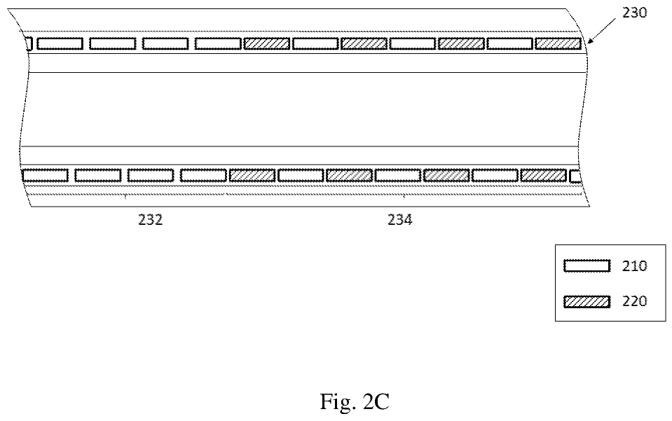

FIG. 2A shows a schematic structural diagram of a combination of two coils according to an exemplary embodiment. FIGS. 2B and 2C show a schematic diagram of a longitudinal cross section including the distal catheter segment of the embodiment in FIG. 2A. FIG. 2B shows a coil made of a round wire, and FIG. 2C shows a coil made of a flat wire. As shown in FIGS. 2B and 2C, the distal catheter segment 110 comprises a coil layer formed from a coil 230 which is formed by combining a first coil 210 and a second coil 220, an inner layer located inside the coil layer and an outer layer located outside the coil layer.

Both the first coil 210 and the second coil 220 comprise a plurality of windings in which inner cavities are formed. The first coil 210 comprises a first region 212 and a second region 214, wherein the first region 212 and the second region 214 respectively comprise a plurality of adjacent windings, the pitches of the first region 212 and the second region 214 may be different, the pitch of the first region 212 is the same as the pitch of the second coil 220, and this pitch is greater than a wire width of the second coil 220.

In the first region 212, the windings of the second coil 220 are interlaced and inserted with the windings of the first coil 210, the windings of the second coil 220 are located between the windings of the first coil 210, and at least some of the windings of the second coil 220 abut against some of the windings of the first coil 210 in the direction of the longitudinal axis, such that the first coil 210 and the second coil 220 are combined at the first region 212 to form the combined coil 230. That is, two portions having different strengths are included in the combined coil 230: a first portion 232 that is the second region 214 of the first coil, and a second portion 234 that is a portion formed by combining the first region 212 of the first coil 210 and the second coil 220.

When the combined coil 230 is subjected to a pushing or pulling force transferred from the proximal catheter segment 106, the windings of the first coil 210 first move under the push or pull force. Since the windings of the second coil 220 are inserted between the windings of the first coil 210, after the windings of the first coil 210 move, at least some of the windings of the second coil 220 are pushed against some of the windings of the first coil 210 and collide with the windings of the first coil 210, thereby achieving movement under the push/pull of the windings of the first coil 210. The force transfer between the windings of the combined coil causes a hysteresis phenomenon when the combined coil deforms under the effect of the push/pull force transferred from the proximal catheter segment 106, reducing the amount of deformation of the combined portion, improving loading amount, namely changing the amount of energy that can be absorbed and accumulated by the spring after loading, and by this way the second portion 234 of the combined coil 230 presents a higher axial tensile strength than an uncombined single coil.

Although FIG. 2A illustrates that the first coil 210 comprises only one first region 212 and one second region 214, it can be understood that the first coil 210 may comprise a plurality of first regions 212 and one or more second regions 214 between and/or outside of these first regions, wherein one second coil 220 is combined in each first region 212 and no second coil is combined in the second regions such that each first region 212 has a tensile strength different from that of the second regions 214.

In an embodiment, the first region 212 of the first coil 210 and each winding of the second coil 220 in the combination have substantially the same inner and outer diameters, and the longitudinal axes of the first coil 210 and the second coil 220 coincide such that an inner cavity and an outer wall of the formed combined coil 230 are not obviously uneven due to disorderly arrangement of the windings.

In the embodiment shown in FIG. 2C, the first coil 210 and the second coil 220 may be two springs made of the same flat wires and having the same diameter, and by screwing the second coil 220 into the first coil 210, a continuous analogous helical disc spring structure is formed. This continuous disc structure increases the radial bearing capacity and axial tensile strength of the coils, thereby increasing the durability of the coils and reducing the risk of coil breakage.

In an embodiment, the first coil 210 and the second coil 220 have the same pitch in the combined first region 212, and the pitch is equal to or more than the sum of the wire widths of the first coil and the second coil 220 such that each winding of the second coil 220 is located between two adjacent windings of the first coil 210.

For example, the first coil 210 and the second coil 220 are both single-strand coils having the same width of wire (or wire width), the pitches of the first region 212 of the first coil 210 and the second coil 220 are both equal to or more than two times the wire width, and the two coils are assembled by means of mutual screwing to obtain a combined coil for replacing a conventional two-strand coil, so that the axial tensile strength, the unwinding resistance and the radial compressive strength of the coil can be improved.

As another example, the first coil 210 is a single-strand coil, the second coil 220 comprises two or more single-strand coils having the same wire width, and in the case where the pitches of the first region 212 of the first coil 210 and the plurality of second coils 220 are both equal to or more than three or more times the wire width of the coils, the first coil 210 and the plurality of second coils 220 may be assembled by means of mutual screwing to obtain a combined coil for replacing a common three-strand or more-strand coil.

In an embodiment, the first coil 210 may extend between the proximal end and the distal end of the catheter body, forming an intermediate layer 114 extending from the proximal catheter segment 106 to the distal catheter segment 110. The first coil 210 may form a taper having a gradually decreasing diameter from the proximal end to the distal end, namely, the diameter of a proximal winding is greater than the diameter of a distal winding, wherein the first region 212 is located only in the distal segment of the first coil 210.

In an embodiment, the first coil 210 and the second coil 220 may be formed by continuous helical springs, the windings of the distal segment and the proximal segment of the formed combined coil 230 respectively have a constant pitch, but the pitch of the distal segment thereof may be different from the pitch of the proximal segment, for example, the pitch of the distal segment is greater than that of the proximal segment. A transition segment may also be comprised between the distal segment and the proximal segment, wherein the coil pitch of the transition segment is not constant but varies gradually in the direction of the longitudinal axis, for example, it gradually increases from the pitch of the proximal segment to the pitch of the distal segment, thereby allowing the shape of the combined coil 230 in the longitudinal axial direction to have no significant abrupt change.

In an embodiment, the pitch of the first region 212 of the first coil 210 is greater than that of the second region 214, and the combined coil 230 formed after the first region 212 of the first coil 210 is combined with the second coil 220 may have substantially the same pitch as the second region 214 of the first coil 210, thereby allowing the shape of the combined coil 230 in the longitudinal axial direction to have no significant abrupt change.

In an embodiment, the number of pitch per inch (PPI) of the second region 214 of the first coil 210 is 80, the PPI of the first region 212 is 40, the PPI of the second coil 220 is 40, and the PPI of the combined region 234 of the first coil 210 and the second coil 220 is 80. The number of pitch per inch (PPI) represents the number of pitches included in the length per inch.

In another embodiment, the number of pitch per inch (PPI) of the second region 214 of the first coil 210 is 80, and the PPI of the first region 212 and the second coil 220 is 26 or less than 26.

In an embodiment, in the combined first region 212, the windings of the first coil 210 and the second coil 220 have an outer diameter range of 0.5588-0.6604 mm and an inner diameter range of 0.4826-0.5842 mm.

In an embodiment, the first coil 210 and the second coil 220 may be made of the same material or different materials. For example, the material of both the first coil 210 and the second coil 220 is 304 stainless steel, or the material of the first coil 210 is 304 stainless steel while the material of the second coil 220 is nickel-titanium alloy.

In the present disclosure, the first coil 210 and the second coil 220 may be coils of the same material and size range as in current catheters, and thus a catheter with better performance than the prior art may be achieved at a production cost comparable to the prior art, thereby solving one or more of the technical problems described above.

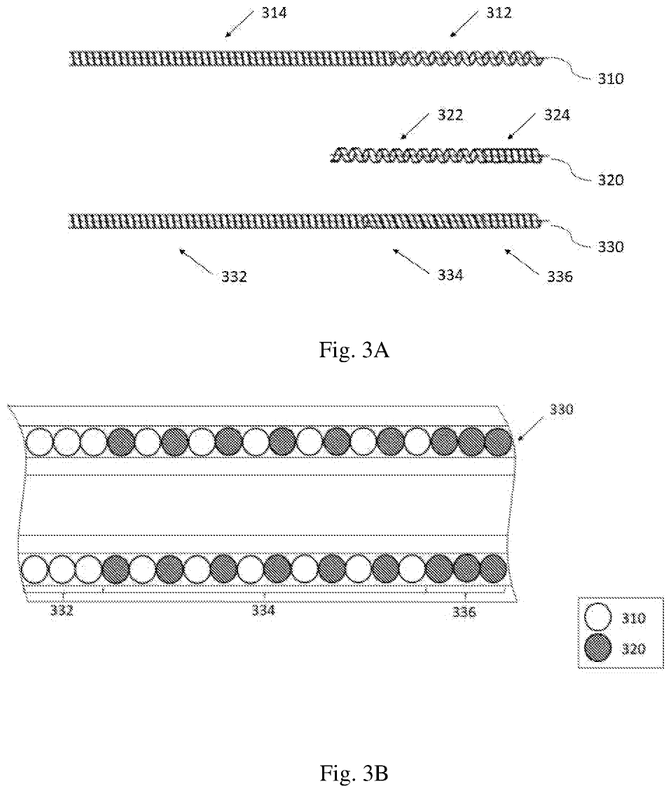

FIG. 3A shows a schematic structural diagram of a combination of two coils according to another exemplary embodiment. FIG. 3B shows a schematic diagram of a longitudinal cross section including the distal catheter segment of the embodiment in FIG. 3A.

In FIGS. 3A and 3B, the vicinity of the distal end of a first coil 310 comprises a first region 312 and a second region 314 adjacent to the first region 312, wherein both the first region 312 and the second region 314 have a plurality of adjacent windings. Unlike that shown in FIG. 1A, a second coil 320 comprises a proximal portion 322 and a distal portion 324, both of which comprise a plurality of adjacent windings and may have different pitches. The proximal portion 322 of the second coil 320 has the same length and pitch as the first region 312 of the first coil 310, and the windings of the proximal portion 322 of the second coil 320 are inserted into the windings in the first region 312 of the first coil such that the first coil 310 and the second coil 320 are combined together. The windings of the distal portion 324 of the second coil 320 extend beyond the first coil 310.

A combined coil 330 formed by the first coil 310 and the second coil 320 comprises three portions: the first portion 332 that is the second region 314 of the first coil 310, the second portion 334 that is a combination of the first region 312 of the first coil 310 and the proximal portion 322 of the second coil 320, and the third portion 336 that is the distal portion 324 of the second coil 320, thereby providing three segments of different tensile strengths.

For example, if the first coil 310 is a spring made of stainless steel and the second coil 320 is a spring made of nickel-titanium alloy, in the combined coil, the tensile strength of the first portion is the tensile strength of the stainless steel spring, the tensile strength of the second portion is the combined tensile strength of the stainless steel spring or the nickel-titanium alloy spring, and the tensile strength of the third portion is the tensile strength of the nickel-titanium alloy spring. Among which the tensile strength of the second portion 334 is the highest, followed by the tensile strength of the first portion 332, and the tensile strength of the third portion 336 is the lowest, so that portions of different spring stiffnesses can be formed at the distal catheter segment 110, wherein the stiffer second portion 334 causes the catheter less likely to deform/unwind under a stress, meanwhile the most distal third portion 336 is kept suitably flexible, providing good operability for the catheter.

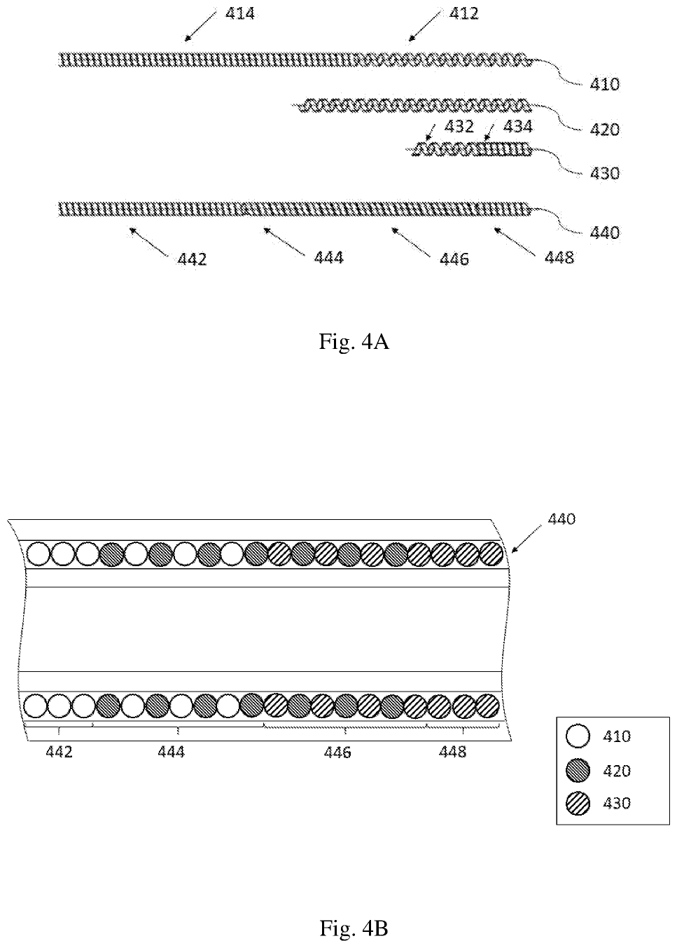

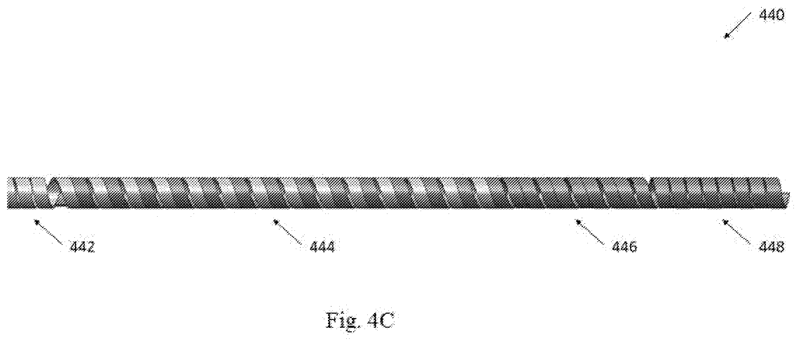

FIG. 4A shows a schematic structural diagram of a combination of three coils according to an exemplary embodiment. FIG. 4B shows a schematic diagram of a longitudinal cross section including the distal catheter segment of the embodiment in FIG. 4A. FIG. 4C shows a three-dimensional perspective schematic diagram of the combined coils of the embodiment in FIG. 4A.

In FIGS. 4A, 4B and 4C, the distal catheter segment 110 is formed by combining three coils. A first coil 410 is similar to the first coil 310 in FIG. 3A, the first coil 410 comprises a first region 412 and a second region 414 that have a plurality of windings, wherein the pitch of the first region 412 is greater than that of the second region 414. The second coil 420 has the same pitch as the first region 412 of the first coil 410 such that the second coil can be inserted into the windings of the first region 412 of the first coil 410.

Unlike that shown in FIG. 3A, the length of the second coil 420 is greater than that of the first region 412, and therefore, after the proximal end of the second coil 420 is screwed into the first coil 410 from the distal end of the first coil 410 until the second coil is completely combined with the first coil 410 in the first region 412, there is still a portion extending beyond the distal end of the first coil 410, near the proximal end of the second coil 420.

A third coil 430, which has two portions of different pitches, with the pitch of the proximal portion 432 the same as the second coil 420, is screwed into the second coil 420 and combined with the portion of the second coil 420 extending beyond the first coil 410. The pitch of the distal portion 434 of the third coil may be less than that of the first portion, which extends beyond the second coil 420.

Thus, a combined coil 440 may comprise four portions: a first portion 442 that is the second region 414 of the first coil 410, a second portion 444 that is a combined portion of the first coil 410 and the second coil 420, a third portion 446 that is a combined portion of the second coil 420 and the third coil 430, and a fourth portion 448 that is a distal portion 434 of the third coil 430, thereby providing at most four segments of different tensile strengths.

For example, if the first coil 410 and the second coil 420 are springs made of a stainless steel material, the third coil 430 is a spring made of nickel-titanium alloy, in the combined coil 440, the tensile strength of the first portion is the tensile strength of the stainless steel spring, the tensile strength of the second portion is the tensile strength of a combination of two stainless steel springs, the tensile strength of the third portion is the tensile strength of a combination of the stainless steel spring and the nickel-titanium alloy spring, and the tensile strength of the fourth portion is the tensile strength of the nickel-titanium alloy spring.

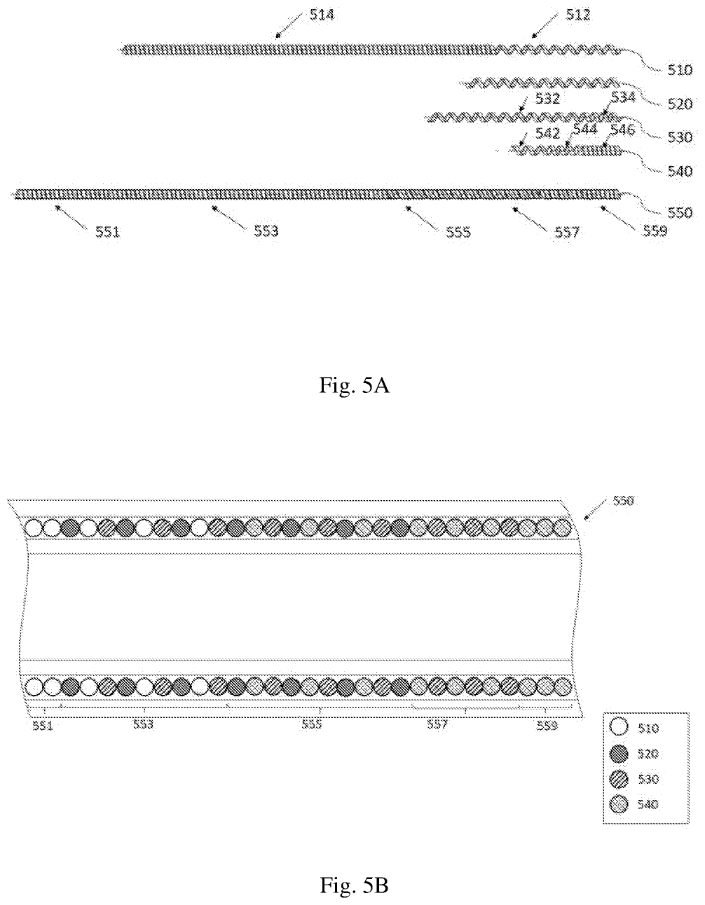

FIG. 5A shows a schematic structural diagram of a distal segment of a combination of four coils according to an exemplary embodiment. FIG. 5B shows a schematic diagram of a longitudinal cross section including the distal catheter segment of the embodiment in FIG. 5A.

In FIG. 5A, the distal catheter segment 110 is formed by combining four coils. A first coil 510 comprises a first region 512 and a second region 514 that have a plurality of windings, wherein the pitches of the first region 512 and the second region 514 are different. A second coil 520 has the same pitch as the first region 512 of the first coil 510, and therefore, the second coil 520 can be screwed into the first coil 510 from the distal end of the first coil 510 to be combined in the first region 512.

The third coil 530 comprises a proximal portion 532 and a distal portion 534 having different pitches, wherein the pitch of the proximal portion 532 is the same as that of the second coil 520, and therefore, the proximal portion 532 of the third coil 530 may be screwed into the second coil 520 from the distal end of the second coil 520 so as to be combined with the second coil 520 and the first coil 510.

A fourth coil 540 comprises a proximal portion 542, a middle portion 544 and a distal portion 546 that have gradually decreasing pitches, wherein the pitch of the proximal portion 542 is the same as that of the proximal portion 532 of the third coil, the pitch of the middle portion 544 is the same as that of the distal portion 534 of the third coil, and therefore, the proximal portion 542 and the middle portion 544 of the fourth coil 540 may be screwed into the third coil from the distal end of the third coil to be combined with the third coil 530 and the second coil 520. The distal portion 546 of the fourth coil 540 extends beyond the third coil 530 without combination with any coil, the distal portion 546 may have a smaller pitch.

The pitch of the first region 512 of the first coil 510 is greater than the sum of the wire widths of the second coil 520 and the third coil 530, such that the second coil 520 and the third coil 530 can be simultaneously screwed into the first region 512.

Windings of the fourth coil 540 are located between the windings of the second coil 520 and the windings of the third coil 530, but not located between the windings of the first coil 510.

A combined coil 550 has five different constituent portions: a first portion 551 that is a second region 514 of the first coil 510; a second portion 553 that is a combined portion of the first region 512 of the first coil 510, the second coil 520 and the proximal portion 532 of the third coil; a third portion 555 that is a combined portion of the second coil 520, the proximal portion 532 of the third coil and the proximal portion 542 of the fourth coil; a fourth portion 557 that is a combined portion of the distal portion 534 of the third coil 530 and the middle portion 544 of the fourth coil; and a fifth portion 559 that is the distal portion 546 of the fourth coil 540. Thus, the combined coil 550 can have five segments of different strengths.

In the above embodiments, the coils shown are single-strand coils, but in other embodiments, any one of the coils may also be a multi-strand coil, namely, a coil formed by winding at least two strands of equal-sized wires. When multi-strand wire coils are used for combination, the pitch of the coils shall be correspondingly configured according to the width of multi-strand wires, and the pitch of the coils used for combination shall be equal to or more than the sum of the wire widths of the coils used for combination so as to screw the coils to form a non-overlapped continuous winding structure.

In the above embodiments, since the catheter body is generally cylindrical, the coils shown are cylindrical helical springs, but in other embodiments, any one of the coils may also be a spring in other shape adapting to the shape of the catheter body.

In the illustration of the exemplary embodiments, the wires for the coils are shown as the round wires or the flat wires. In some preferred embodiments, the wires for the coils are the flat wires, but are not limited thereto, and the wires for the coils may also be wires in other shapes. The materials for the coils may be selected as required, and the materials of different coils may be the same or different to achieve different flexibilities or to meet different tensile strength requirements.

The above embodiments in which a plurality of coils are combined are merely exemplary embodiments of the present disclosure. In actual use, those skilled in the art may combine different numbers of coils in different regions with different pitches as required to achieve a coil layer having a desired strength in a given region.

The inventors have measured the spring stiffnesses of a single coil and a combined coil under the condition that the single coil has the same or similar parameters (including inner diameter, outer diameter, cross-sectional area, material properties). The measurement results are shown in Table I.

TABLE I
Comparison of spring stiffnesses of different coils

| | | | | Combined |
|---|---|---|---|---|
| | Single- | Single- | | coil of two |
| | strand | strand | 14-strand | single-strand |
| | coil 1 | coil 2 | coil | coils |
| Loading capacity (pounds | 2318 | 6321 | 35475 | 7432 |
| per square inch) | | | | |
| Amount of deformation | 213.66 | 515.55 | 385.85 | 51.26 |
| (%) | | | | |
| Spring stiffness (Loading | 1085 | 1226 | 9194 | 14498 |
| capacity/Amount of | | | | |
| deformation) | | | | |

From Table I, it can be seen that the spring stiffness of the combined coil formed by two single-strand coils is much greater than that of single-strand coils or multi-strand coils (more than 10 times the spring stiffness of single-strand coils or 1.5 times the spring stiffness of 14-strand coils) under the condition of similar other parameters. It can be seen therefrom that when the combined coil is subjected to a load, the amount of deformation is reduced due to the hysteresis effect, thereby increasing the load for deformation per unit, namely, increasing the spring stiffness.

In the present disclosure, a novel coil in a spiral shape is obtained by spirally combining two or more single-strand coils with one another, and multi-strand coils are replaced with such a combination. The axial tensile strength and radial compressive strength of the combined coil are improved due to mutual contact of the coils that are pushed against each other, and the capability to resist unwinding in an axial direction is also enhanced, thereby improving bearing capacity, reliability, firmness and durability of the catheter.

In this description, the “axial tensile strength” of the coil may be represented by the spring stiffness of the coil, wherein the spring stiffness is the ratio of the loading capacity to the amount of deformation of the spring or a load required for deformation per unit.

The present disclosure further provides a method for manufacturing an elongated catheter, wherein the elongated catheter has a longitudinal axis extending between a proximal end and a distal end and has a lumen or cavity. The method comprises: arranging a first end of a first coil near the proximal end of the catheter, and arranging a second end of the first coil near the distal end of the catheter; providing a second coil; and positioning at least some of windings of the second coil in a first region of the first coil including a plurality of adjacent coils by screwing a winding at one end of the second coil into the first coil along the longitudinal axis from the second end of the first coil.

In an embodiment, the length of the first coil is greater than that of the second coil, and the first coil comprises at least one second region in which there is no inserted winding of the second coil.

In an embodiment, in windings of a combined first region of the first coil and a plurality of second coils, the windings of the first coil and the second coil are connected at some positions to prevent possible offset or displacement of two screwed ends of the coils. For example, a first coil winding and a second coil winding abutting against each other are subjected to a partial spot welding operation by means of laser welding at two ends and a middle part or other suitable parts, so as to ensure that outlines of a plurality of coils after screwing are concentrically overlapped, thereby obtaining stable uniform outer and inner diameters and reducing the risk of the first coil and the second coil being separated from each other under a stress.

The first coil may be a spring layer extending from the proximal end to the distal end of the catheter, the second coil is combined with the first coil in a certain region of the distal segment of the first coil to enhance the axial tensile strength and radial bending strength of the specific region, and the second coil does not extend to the proximal segment of the catheter.

A catheter using the combined coil in the present disclosure as a spring layer may be used for percutaneous insertion into a blood vessel to support and guide a guide wire when passing through a locally stenotic lesion of the blood vessel so as to prevent the phenomenon that the guide wire cannot pass through the stenotic lesion, or used for exchange with another guide wire. For example, the catheter may be used as an instrument for a percutaneous coronary interventional therapy.

The above embodiments are described herein by way of examples only. Many variations are possible without departing from the scope of the present disclosure as defined in the claims. Although various examples and other information are used to explain various aspects within the scope of the appended claims, specific functions or configurations in such examples should not be used as limitations to the claims, as those skilled in the art will be able to use the examples of these claims to derive various implementations.

Furthermore, although some subject matter may be described herein in illustrative languages of specific structural features and/or method steps, it should be understood that the subject matter defined in the claims is not necessarily limited to the described features or actions. For example, such functions may be differently distributed or executed in components other than those marked herein. The described features and steps are disclosed only as examples of components of the system and method within the scope of the appended claims.

1. A catheter, comprising:
a tubular elongated body, wherein the tubular elongated body extends along a longitudinal axis between a proximal end and a distal end, and the tubular elongated body has a lumen;
wherein, at least a portion of the tubular elongated body is defined by a first coil, and the first coil comprises a plurality of windings extending along and around the longitudinal axis,
wherein, the first coil comprises at least one first region arranged near the distal end of the tubular elongated body and including a plurality of adjacent windings, the first region has a plurality of windings inserted from at least one second coil therein, and the inserted windings of the second coil are positioned between the windings of the first coil; and
the first coil further comprises at least one second region including a plurality of adjacent windings, wherein the second region does not have windings inserted from the second coil.

2. The catheter of claim 1, wherein in the first region, at least some of the windings of the second coil abut against some of the windings of the first coil.

3. The catheter of claim 2, wherein when the catheter is pushed or pulled along the longitudinal axis of the tubular elongated body, at least some of the windings of the second coil are configured to be pushed against some of the windings of the first coil in the first region.

4. The catheter of claim 1, wherein at the first region, the windings of the second coil have substantially the same diameter as the windings of the first coil.

5. The catheter of claim 1, wherein the windings of the at least one second coil are inserted between the windings of the first coil by screwing the second coil into the first coil along the longitudinal axis.

6. The catheter of claims 1-5, wherein the first region is disposed at a predetermined position of the first coil along the longitudinal axis to provide the tubular elongated body with an axially varying strength distribution.

7. The catheter of claim 1, further comprising a third coil having a plurality of windings,
wherein some of the windings of the third coil are located between the windings of the second coil and are not located between the windings of the first coil.

8. The catheter of any one of claims 1-7, wherein a wire for any one of the coils is a flat wire.

9. The catheter of any one of claims 1-8, wherein any one of the coils is a helical spring.

10. The catheter of any one of claims 1-9, wherein any one of the coils is a single-strand wire spring or a multi-strand wire spring.

11. The catheter of any one of claims 1-10, wherein the first coil and the second coil are made of the same material.

12. The catheter of any one of claims 1-10, wherein the first coil and the second coil are made of different materials.

13. The catheter of claim 1, wherein the pitch of the first coil undergoes gradual transition along the longitudinal axis from a first number of pitch per inch at the proximal end to a second number of pitch per inch.

14. The catheter of claim 13, wherein the first number of pitch per inch is 80, and the second number of pitch per inch is 40 or 26 or less than 26.

15. The catheter of claim 14, wherein the number of pitch per inch of the second coil is 40 or 26 or less than 26.

16. The catheter of any one of the preceding claims, wherein at the first region, the first coil and the second coil have an outer diameter range of 0.5588-0.6604 mm and an inner diameter range of 0.4826-0.5842 mm.

17. A method for manufacturing an elongated catheter, the elongated catheter having a longitudinal axis extending between a proximal end and a distal end and having a lumen, the method comprising:
arranging a first end of a first coil near the proximal end of the catheter, and arranging a second end of the first coil near the distal end of the catheter;
providing a second coil; and
positioning at least some of windings of the second coil in a region of the first coil including a plurality of adjacent coils by screwing a winding at one end of the second coil into the first coil along the longitudinal axis from the second end of the first coil.
